(19) United States
(12) Patent Application Publication
(10) Pub. No.: US 2020/0008456 A1
(43) Pub. Date: Jan. 9, 2020

(54) Snacks, Beverages, And Edible Additives With Sun Defense Nutriments And Methods Of Manufacture Thereof

(71) Applicants: John Shannon Kenner, Fernandina Beach, FL (US); Kathryn Johnston Kenner, Fernandina Beach, FL (US)

(72) Inventors: John Shannon Kenner, Fernandina Beach, FL (US); Kathryn Johnston Kenner, Fernandina Beach, FL (US)

(21) Appl. No.: 16/571,044

(22) Filed: Sep. 13, 2019

Related U.S. Application Data
(63) Continuation-in-part of application No. 15/608,917, filed on May 30, 2017, now abandoned.

(51) Int. Cl. A23L 33/155; A23L 25/00; A23L 33/16; A61K 36/05; A61K 31/375; A61K 31/355; A61K 35/748; A61K 31/07; A61K 31/59; A61K 33/00

(52) U.S. Cl. CPC A23L 33/155; A23L 25/00; A23L 33/16; A61K 36/05; A61K 31/375; A23V 2002/00; A61K 35/748; A61K 31/07; A61K 31/59; A61K 33/00; A61K 31/355

(57) ABSTRACT

The present disclosure provides generally for snacks with sun defense nutriments that may allow participants to safely engage in sun activities and methods related to manufacturing sun defense nutriments. Sun defense nutriments may comprise sun defense attributes that may provide sun activity protection when the sun defense nutriment is ingested. Snacks with sun defense nutriments may be customized to provide effective protection measures for a particular sun activity. In some aspects, methods of manufacturing may allow for the generation of combinations of sun defense nutriments for production of snacks. In some implementations, the combinations may be generated based on one or more criterion related to sun defense attributes, sun activities, flavors, or nutriment types, as non-limiting examples.

### CROSS-REFERENCE TO RELATED APPLICATIONS

This application claims priority to and the full benefit of U.S. Non-Provisional patent application Ser. No. 15/608,917, filed May 30, 2017, and titled “SUN DEFENSE NUTRIMENTS AND METHODS OF MANUFACTURE THEREOF”, which further claims priority to U.S. Provisional Patent Application Ser. No. 62/344,206, filed Jun. 1, 2016, and titled “SUN DEFENSE NUTRIMENTS AND METHODS OF MANUFACTURE THEREOF”, the entire contents of which are incorporated herein by reference.

### BACKGROUND OF THE DISCLOSURE

Traditionally, human beings would be in the sun for hours on end without recognizing the potential detrimental effects that this prolonged exposure to sunlight might have on them. Once some of these effects became known, human beings began using shade to avoid unwanted, prolonged, or dangerous exposure to the sun. They then developed portable versions of this concept, such as umbrellas, hats, or visors. Other articles of clothing were also used to avoid overexposure.

Eventually, human beings started applying sunscreen to minimize the effects of overexposure to the sun. This is done to protect themselves from the sun's negative effects, which range from irritated skin and peeling to skin cancer, that previous solutions could not provide. These sunscreens are typically available in different strengths, depending on need or expected time in the sun. As time went on, different applications for sun protection arose, such as sunscreen application in different forms, such as a spray or oil. These help mitigate short-term sun damage, but studies indicate that extended use may have adverse effects and limited effectiveness against deep tissue damage.

Current non-topical forms of sun defense include vitamins or supplements, which are generally intended for long-term effectiveness. Further, as the general population has gravitated toward diets containing significant amounts of chemicals, genetically-modified ingredients, and processed sugars and grains, the human body's natural sun defense mechanisms have worked less effectively. Regardless of the potential negative effects of sun or its synthetic equivalent, one thing remains clear: human beings are still active in the sun, and products should be created to foster and encourage a safe way to continue to do so.

### SUMMARY OF THE DISCLOSURE

What is needed is a snack with sun defense nutriments that incorporates multiple forms of defense depending on the activity a person is about to engage in, with sun defense attributes as a base to provide a minimum layer of protection before engaging in these activities. This sun defense nutriment can come in the form of several embodiments, ranging from, but not limited to, a nutrient bar, a chew, a beverage, or something that mixes with other food or preparatory materials. Regular use of these snacks will allow a user to more effectively fight against sunburn and long-term skin damage. This also means less reliance on sunscreens, some of which have ingredients that are considered unsafe and ineffective against long-term skin damage.

These snacks with sun defense nutriments work in conjunction with the human body's own defense system, including those that remove and neutralize free radicals, to fight sun damage while not introducing other risks, like toxic chemicals such as petrochemicals, to accomplish this goal. The snacks would also aid the body in seeking to remove and neutralize free radicals produced by sun exposure. By being an ingestible form of sun defense for various sun-related activities, this also has the benefit of working constantly, ideally providing a consistent boost in sun defense protection depending on intake or how often someone used a nutriment. This added benefit and accessibility helps to protect the body from the 70% of sun exposure that occurs as part of daily life and when not wearing sunscreen.

The present disclosure provides generally for snacks with sun defense nutriments that may allow participants to safely engage in sun activities. According to the present disclosure, sun defense nutriments within the snacks may comprise sun defense attributes that may provide sun activity protection when the snack is ingested. In some embodiments, sun defense nutriments may be customized to provide effective protection measures for a particular sun activity.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1A illustrates exemplary sun defense nutriments.

FIG. 1B illustrates an exemplary assortment of sun defense nutriments.

FIG. 1C illustrates an exemplary assortment of quick release sun defense nutriments.

FIG. 1D illustrates alternate exemplary sun defense nutriments.

FIG. 2A illustrates exemplary liquid sun defense nutriments, according to some embodiments of the present disclosure.

FIG. 2B illustrates exemplary liquid sun defense nutriments, according to some embodiments of the present disclosure.

FIG. 2C illustrates exemplary liquid sun defense nutriments, according to some embodiments of the present disclosure.

FIG. 2D illustrates exemplary liquid sun defense nutriments, according to some embodiments of the present disclosure.

FIG. 3 illustrates an exemplary spice grinder that may integrate sun defense nutriments into ground spices.

FIG. 4 illustrates an exemplary beverage container with filtering lid.

FIG. 5A illustrates an exemplary process step for adding a sun nutriment powder to a liquid utilizing a water bottle cap, according to some implementations of the present disclosure.

FIG. 5B illustrates an exemplary process step for adding a sun nutriment powder to a liquid utilizing a water bottle cap, according to some implementations of the present disclosure.

FIG. 5C illustrates an exemplary process step for adding a sun nutriment powder to a liquid utilizing a water bottle cap, according to some implementations of the present disclosure.

FIG. 5D illustrates an exemplary process step for adding a sun nutriment powder to a liquid utilizing a water bottle cap, according to some implementations of the present disclosure.

FIG. 6 illustrates exemplary method steps for developing an effective sun defense nutriment for a sun activity.

FIG. 7 illustrates exemplary method steps for providing sun defense nutriments for an event with one or more sun activities.

FIG. 8 illustrates exemplary method steps for developing a sun defense nutriment.

FIG. 9 illustrates an exemplary network system that may be used to implement aspects of the present disclosure including executable software.

FIG. 10A illustrates an exemplary application interface for a sun defense nutriment planner that may be used to implement aspects of the present disclosure.

FIG. 10B illustrates an alternate exemplary application interface for a sun defense nutriment planner that may be used to implement aspects of the present disclosure.

FIG. 11 illustrates an exemplary application interface for a sun defense nutriment planner that may be used to implement aspects of the present disclosure.

FIG. 12 illustrates an exemplary application interface for a sun defense nutriment planner that may be used to implement aspects of the present disclosure.

### DETAILED DESCRIPTION

The present disclosure provides generally for sun defense nutriments that may allow participants to safely engage in sun activities. According to the present disclosure, sun defense nutriments may comprise sun defense attributes that may provide sun activity protection when the sun defense nutriment is ingested. In some embodiments, sun defense nutriments may be customized to provide effective protection measures for a particular sun activity.

In the following sections, detailed descriptions of examples and methods of the disclosure will be given. The description of both preferred and alternative examples though thorough are exemplary only, and it is understood that to those skilled in the art that variations, modifications, and alterations may be apparent. It is therefore to be understood that the examples do not limit the broadness of the aspects of the underlying disclosure as defined by the claims.

### Glossary

Snack: as used herein refers to any convenient ingestible food or beverage. In some aspects, the snack may be used to enhance another food or beverage, such as a beverage enhancer or topping for a yogurt or cottage cheese. As non-limiting examples, a snack may comprise bars, chews, chips, cookies, candy, gum, or crumbles. Other non-limiting examples may include a juice, shake, smoothie, smoothie mix, or flavored water.
Sun Activity: as used herein refers to any activity that may cause an exposure to the sun or synthetic light with characteristics of sunlight, such as UV rays and heat, or any activity that increases risk of unhealthy condition, such as exhaustion, dehydration, or overheating, as non-limiting examples A sun activity may occur outdoors or in an in-door or enclosed area with direct or indirect exposure to sunlight, such as a greenhouse or an open-air arena.
Sun Defense Attribute: as used herein refers to any attribute of a sun defense nutriment that may provide protection against a sun activity side effect. In some aspects, a sun defense attribute may comprise a vitamin, such as Vitamin A, Vitamin B, Vitamin C, Vitamin D, and Vitamin E, as non-limiting examples. In some embodiments, a sun defense nutriment may comprise ingredients that may release odors or oils, wherein the odors and oils may be the sun defense attributes, potentially repelling insects or retaining moisture on the skin surface. For example, the sun defense nutriment may comprise garlic, the sun activity side effect may comprise insect bites, and ingestion of the snack may release oils with insect repelling odors, which may be the sun defense attribute of garlic. In some implementations, a sun defense nutriment may comprise multiple sun defense attributes, which may protect against the same or different sun activity side effects.
Sun Defense Nutriment: as used herein refers to any ingredient of a sun defense nutriment that provides a sun defense attribute. As non-limiting examples, a sun defense nutriment may be a particular vitamin, an extract, a natural or synthetic derivative, an amino acid, a fruit, or a vegetable.
Sun Defense Nutriment Planner: as used herein refers to any device, mechanism, or process that may assess one or more sun activities to determine associated snacks and sun defense nutriments that may provide protection against one or more sun activity side effects associated with the sun activities.
Sun Activity Side Effect: as used herein refers to any potential damaging effects that may be caused by participating in a sun activity. A sun activity side effect may be caused by exposure to particular elements, such as, for example, sunlight, heat, wind, or precipitation. A sun activity side effect may be caused by the activity itself, which may be exacerbated by the elements. For example, the sun activity may be an intense sport that may cause muscle strain and dehydration, which may be worsened by heat or made more prone to injury by a wet field. Potential damaging effects may include, for example, sun burn, dehydration, sea sickness, allergies, sinus onset or aggravation, or insect bites.
Sun Defense Nutriment: as used herein refers to any ingestible ingredient that may be a component of an edible snack, beverage, or additive, including, for example, food, drink, food additive, ingredient, or condiment, that possesses one or more sun defense attribute. In some embodiments, a sun defense nutriment may be used in conjunction with other edible components.

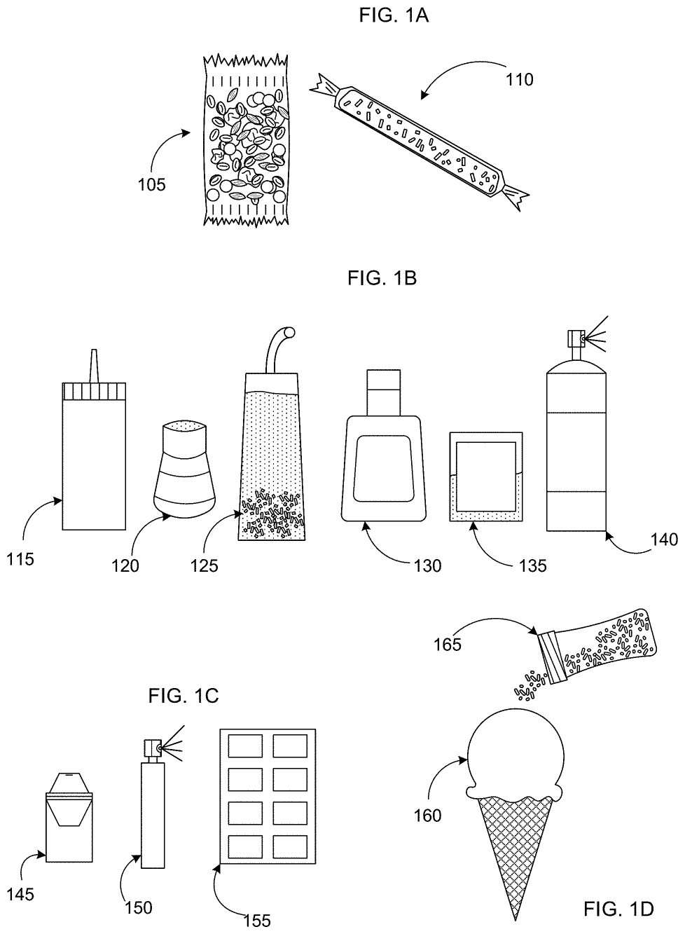

Referring now to FIG. 1A, exemplary snacks are illustrated. In some aspects, a snack may comprise a bar 110, which may comprise a mix of sun defense nutriments and other ingredients. In some embodiments, the other ingredients may include general health-conscious ingredients, such as, for example, granola, natural flavorings, or seasonings. Other ingredients may include caramel, nougat, Nutella, or marshmallow. In some aspects, sun defense nutriments may be combined to create popular snack flavor profiles, such as, for example, sweet, salty, sweet and salty, savory, and spicy.

Each snack may comprise a combination of sun defense nutriments to effectively combat predefined sun activity side effects, wherein none of the sun defense nutriments or other ingredients may counteract those protections or if they do, there is a sufficient balance to offset that effect. For example, where the sun activity side effect may comprise dehydration, none of the sun defense nutriments or other ingredients of the same snack may increase likelihood of dehydration.

In some embodiments, a snack may comprise a bag of edible components 105 wherein one or more of the components comprise sun defense nutriments. For example, the bag of edible components 105 may comprise granola bites, trail mix, popcorn, cookies, or chips. In some aspects, only portions of the edible components may comprise sun defense nutriments, such as, for example, in a trail mix, wherein sun defense nutriments may comprise dried cherries, chocolate pieces, salt, peanut butter bites, and sunflower seeds and other ingredients may comprise dried marshmallows, granola, and pretzels. In some embodiments, the granola may be treated with sun defense nutriments, such as by coating it in coconut oil.

As non-limiting examples, sun defense nutriments may comprise organic peanut butter, dark chocolate chips (unsweetened chocolate, cane sugar), organic clover honey, cacao butter, cherries, pea protein, organic whole grain quinoa, organic almond butter, organic flaxseeds, chia seeds, organic sunflower seeds, flax seeds, sunflower lecithin, green tea, Spirulina, chlorella, sea salt, garlic, or organic coconut oil. As non-limiting examples, sun defense attributes may comprise Vitamin C, Vitamin E, Vitamin A, antioxidants, Vitamin D, chlorophyll, glutathione, phytonutrients, carotenoids, astaxanthin, polyphenols, alpha-linolenic fatty acids, protein, omega-3 fatty acids, magnesium, B-vitamins, potassium, calcium, flavonoids, biotin, copper, niacin, folate, manganese, medium-chain fatty acids, and medium-chain triglycerides (aka MCTs). In some aspects, snacks may comprise combinations of the above exemplary sun defense nutriments and sun defense attributes. In some aspects, a snack may comprise one or more seed type, nut butter type, and antioxidant, such as fat soluble or water soluble.

In some implementations, sun defense nutriments may be integrated throughout the edible components, such as, for example, where the bag of edible components 105 comprises chips, the sun defense nutriments may comprise the base, such as beets or carrots, as well as part of a spice and flavor mix in which the chips may be coated. For example, banana (Vitamin B6) and carrot slices (high in Vitamin A) may be cooked in nutrient-rich sunflower oils and then tossed in a coating of pink Himalayan salt (for electrolytes), dried green tea, and powdered chili peppers. Overall, the chips may protect against skin damage, risk of skin cancer, and dehydration, all of which may be sun activity side effects where the sun activity may occur outside. For example, the sun activity may comprise a day at the beach or a day of skiing.

In some aspects, snacks may be developed for a particular participant, such as by age group, athletic level, or nutrition requirements. For example, a snack may include sun defense nutriments that may be particularly beneficial for a small child, including, for example, balanced levels of calcium, iron, folate, and fiber. As another example, a snack may comprise sun defense nutriments particularly beneficial to a high performing athlete who may have increased metabolism or increased energy needs. As another example, a participant may have diabetes, and the snack may allow for a slower digestion of the sugars, which may limit risk of an insulin spike. For example, the snack may be high in fiber and low in simple sugars.

Referring now to FIG. 1B, an exemplary assortment of snacks are illustrated, wherein the snacks may comprise enhancing components that may be added to standard food and recipes to transform the standard food into a snack with sun defense nutriments. For example, snacks may comprise condiments 115, spices 120, oils 125, sauces or dressings 130, powdered food mixes 135 such as for soups, brines, or dressings, or pan sprays 140. For example, the pan spray 140 may comprise the sun defense nutriments of coconut oil and garlic, wherein the pan spray 140 may effectively coat foods cooked in the pan enhancing any food into a quick energy boost combined with a defense against and insect bites.

Referring now to FIG. 1C, an exemplary assortment of quick release snacks are illustrated. In some aspects, a participant may desire an immediate or quick-release effect from the sun defense nutriments. The speed of the effect may be related to the ease of digestion, absorption properties, and other factors. In some embodiments, the snacks may comprise dissolving tabs 145, mouth spray 150, or chewable tablets 155, such as gum, gummies, lollipops, or other candies. In some aspects, the snack may comprise sun defense nutriments of varying speeds of digestion, which may allow for prolonged protection against sun activity side effects.

Referring now to FIG. 1D, exemplary snacks are illustrated. In some embodiments, sun defense nutriments may comprise different sun defense attributes, wherein a participant may be able to combine sun defense nutriments to develop complex sun activity solutions. For example, where the sun activity exposes a participant to heat, sun, and insects, one snack may comprise ice cream 160 with sun protection and hydration as the sun defense attributes, and an additional sun defense nutriment may be integrated into sprinkles 165 with sun protection and insect repellant as the sun defense attributes. Other variations on toppings may include, but would not be limited to, chocolate, gummy candies, or fruit, which may comprise sun defense attributes. The sprinkles 165 may be added to the ice cream 160 to create a snack with three sun defense attributes of sun protection, hydration, and insect repellant. Further, the ice cream 160 may release protective properties that increase sun defense attributes as it melts or as it mixes with other ingredients, such as the sprinkles 165.

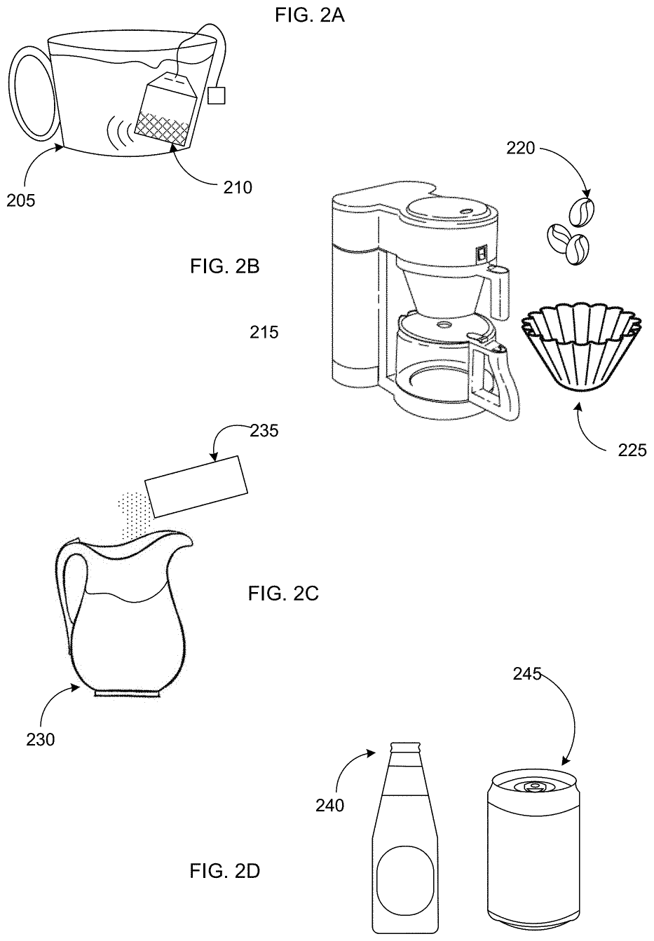

Referring now to FIGS. 2A-2D, exemplary liquid snacks with sun defense nutriments are illustrated. In some aspects, as is illustrated in FIG. 2A, a liquid snack may comprise a steeped beverage 205, such as tea, wherein the tea bag 210 may comprise one or more sun defense nutriments that may imbue the liquid with sun defense attributes during the steeping process. Similarly, machine-made beverages may comprise sun defense nutriments. For example, a typical coffee maker 215 may use coffee beans 220 and a filter 225, wherein one or both of the coffee beans 220 and the filter 225 may comprise sun defense nutriments that may imbue brewed coffee with sun defense attributes.

In some embodiments, such as illustrated in FIG. 2C, a packet 235 of powdered sun defense nutriments may be added to a pitcher 230 or other container of liquid, wherein the addition of the powder may imbue the liquid with one or more sun defense attributes. Though illustrated as a packet 235 of powdered sun defense nutriment for multiple servings, other examples may include a container of sun defense nutriment for liquids may comprise a bottle of liquid concentrate, a box of dissolving tablets, or single-serving packets of powdered concentrate.

In some aspects, the liquid may comprise a sun defense nutriment prior to the addition of the powder, which may allow for permutations of sun defense attributes. For example, the liquid may comprise water with electrolytes, wherein the sun defense attributes of the liquid may be hydration and energy. Adding the powder may provide the additional sun defense attribute of a cooling effect, which may protect against the sun activity side effect of overheating. The combination may be useful in sun activities that expose participants to high heat situations, such as a marathon, hiking, or sports game.

In some aspects, such as illustrated in FIG. 2D, a snack with sun defense nutriments may comprise ready-made beverages in bottles 240 or cans 245. For example, a hard cider or beer with sun defense attributes may be convenient sun defense nutriments for a tailgating event. Other examples may include soda, juices, energy drinks, water, wine, or liquor.

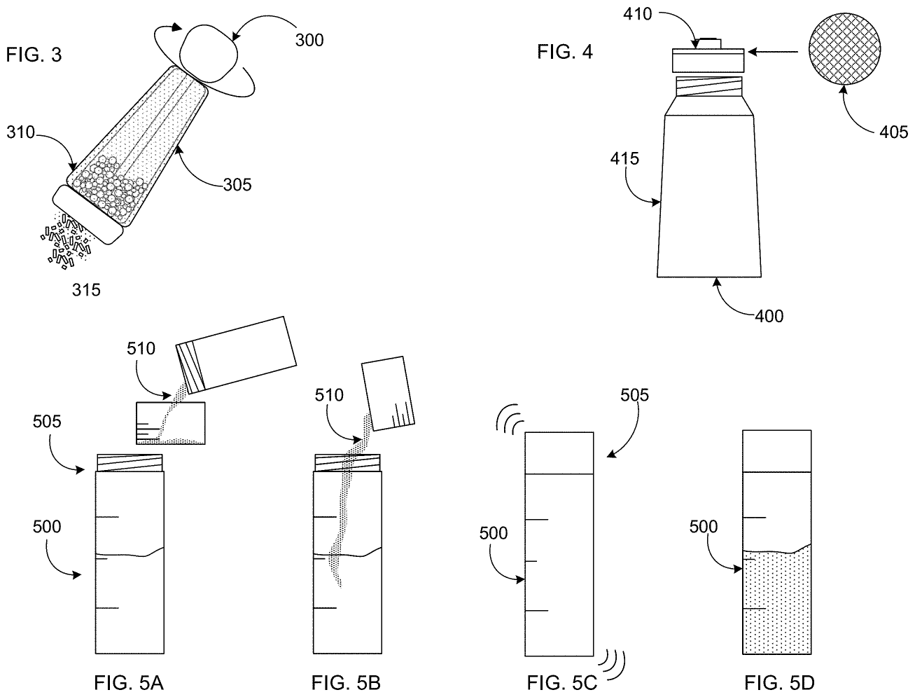

Referring now to FIG. 3, an exemplary spice grinder 300 that may integrate sun defense nutriments 310 into ground spices 315 is illustrated. In some implementations, a spice grinder 300 may comprise a separate refillable pocket or layer that may hold sun defense nutriments 310, wherein the grinding of the contained spice 305 may proportionally integrate sun defense nutriments 310 into the contained spice 305 resulting in a mixed seasoning of ground spices 315 with sun defense attributes.

Referring now to FIG. 4, an exemplary beverage container 400 with filtering lid 410 is illustrated. In some implementations, the filtering lid 410 may comprise a filter slot 415, wherein a filter 405 may be inserted for use. The filter 405 may comprise sun defense nutriments that may imbue liquid that passes through the filtering lid 410 with sun defense attributes. In some embodiments, the filtering lid 410 may comprise a filter strength indicator (not shown), which may indicate to a participant whether the filter 405 is still effectively adding sun defense attributes to liquid. In some embodiments, the filtering lid 410 may comprise a filter strength module or control (not shown) that may allow a participant to determine dosage, strength, or quantity of sun defense attribute to be added. This control (not shown) may also be programmable or have a time release feature to dispense sun defense attributes over a set period of time. The filter 405 may be removed and exchanged for a variety of reasons, such as a loss of effectiveness, age, or a need for different sun defense attributes. In some aspects, the filter 405 may add flavor to filtered liquid.

Referring now to FIGS. 5A-5D, exemplary process steps for adding a sun nutriment powder 510 to a liquid utilizing a water bottle cap 505 is illustrated, wherein the water bottle cap 505 comprises measuring indicators in conjunction with the water bottle 500 to allow for convenient measuring. As illustrated in FIG. 5A, sun defense nutriment powder 510 may be added to a water bottle cap 505 fill line commensurate with the fill line of the liquid in the water bottle 500. As illustrated in FIG. 5B, the sun defense nutriment powder 510 may be added to the liquid. As illustrated in FIG. 5C, the water bottle cap 505 may be placed on the water bottle 500, and the water bottle 500 may be shaken. As illustrated in FIG. 5D, the shaking of the water bottle 500 may evenly disperse the sun defense nutriment throughout the liquid. In another embodiment, not shown, a water bottle cap may also include a time release feature to deliver a sun nutriment.

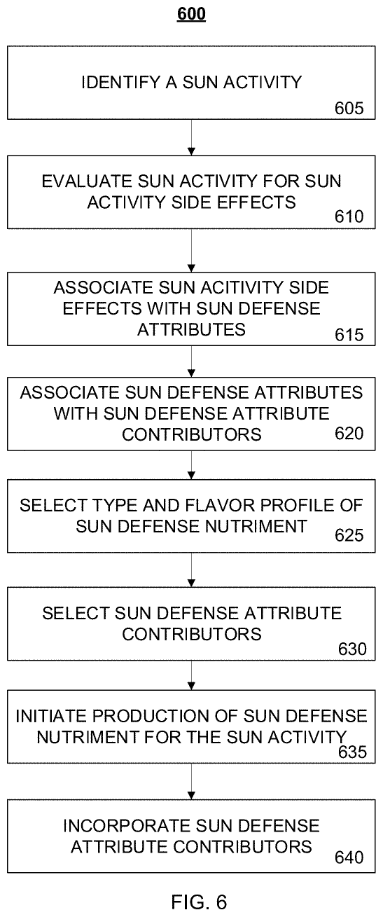

Referring now to FIG. 6, an exemplary method 600 for developing a snack with effective sun defense nutriments for a sun activity is illustrated. At 605, a sun activity may be identified. At 610, the identified sun activity may be evaluated for sun activity side effects. At 615, sun defense attributes may be associated with the sun activity side effects. At 620, the sun defense attributes may be associated with sun defense nutriments.

At 625, a type and flavor profile of sun defense nutriments may be selected. The types selected at 625 may be based on one or more factors. These factors may include, for example, the type that may be based on convenience for use during the sun activity, preparation level preference of a participant, or spoliation requirements of the sun activity.

The flavor profiles of the sun defense nutriments selected may be based on a number of factors, including for example, popular flavors associated with the type of sun defense nutriment or typical cravings associated with the sun activity side effects. For example, fruit flavors may be preferable for gummy candies but not for a brine, and salty flavors may be preferable for chips but not for a beverage. In some embodiments, the flavor profile may support the sun defense attributes. For example, a sun defense nutriment that provides hydration may have a refreshing flavor profile, whereas a sun defense nutriment that provides energy may have a green tea flavor profile as green tea may generally be associated with energization.

At 630, sun defense nutriments may be selected from those associated with the sun defense attributes at 620 and may be based on the type and flavor profile of the sun defense nutriment. For example, a sun defense nutriment that may add a floral scent may not be appropriate for a granola bar, and a sun defense nutriment that may lose its potency at high or low temperatures may not be appropriate for a soup mix or a popsicle.

At 635, the production of the snack with sun defense nutriments may be initiated, and at 640, the selected sun defense nutriments may be incorporated into the snack. The method of incorporation may vary depending on the type of snack and the sun defense nutriment. For example, where the snack may be a trail mix and the sun defense nutriment may comprise a sunflower seed, the incorporation may involve adding shelled sunflower seeds to the trail mix.

As another example, the snack may be a tea, and the sun defense nutriment may be hibiscus, açai berry, and vitamin D. The incorporation may involve treating one or both the hibiscus and açai berries with vitamin D, drying the hibiscus and açai berry, and integrating the dried hibiscus and açai berries into a tea bag with other tea components.

In some embodiments, the sun defense nutriment may comprise CoQ10, which may be integrated into snacks to add one or more sun defense attributes. For example, CoQ10 may provide sun defense attributes that may reduce side effects associated with migraines, treat muscle breakdown, reduce or prevent sun damage, or provide energy. In some forms, CoQ10 may be lipid-soluble and may be suited for solid or non-liquid snacks, as a method to allow for higher bioavailability. In some aspects, CoQ10 may integrated as oil suspension or lipid microspheres within snacks. For example, the oils may comprise soybean or olive oil, and the snack may comprise a salad dressing, chips, or gummies.

In some aspects, high temperatures, such as caused by cooking or frying, may reduce some beneficial properties of CoQ10. Accordingly, components that may reduce or limit temperature exposure may be integrated around the lipid microspheres in sun defense nutriments that may be exposed to higher temperatures, such as for pan sprays. Similarly, sources with lower smoke points, such as grapeseed oil, may withstand higher temperature with limited reduction in effectiveness.

In some embodiments, the sun defense nutriment may comprise idebenone, which is a synthetic comprising qualities similar to CoQ10 antioxidant properties. Further, idebenone may help with memory.

In some embodiments, the sun defense nutriment may comprise vitamin A, which may help with immune system maintenance, vision (particularly low light vision), skin and cellular health, and bone metabolism. Vitamin A may be directly integrated into sun defense nutriments or indirectly through natural sources of vitamin A, including, for example, carrots, kale, pumpkin, egg, apricot, papaya, or mango.

In some embodiments, the sun defense nutriment may comprise vitamin E, which may be an effective antioxidant. Vitamin E may be useful for treating and preventing heart diseases, increasing energy, improving physical endurance, and treating diseases of the brain and nervous system, such as dementias, epilepsy, or night cramps. Common natural sources of Vitamin E may include oils such as sunflower, wheat germ, or almond; green, leafy vegetables; and fruits such as mango, papaya, or kiwi. As illustrative examples, Vitamin E may be integrated into snacks such as bars, beverages, sauces, or cooking oils.

In some embodiments, the sun defense nutriment may comprise beta-carotene, which may have increased bioavailability when integrated into non-liquid snacks. In some aspects, beta-carotene may be used as a source for sunless tanning, which may be useful in conjunction with sun defense to discourage the need for unprotected sun exposure. In some embodiments, the sun defense nutriment may comprise vitamin B3.

In some embodiments, the sun defense nutriment may comprise vitamin C, which may serve as an antioxidant or as support to the immune system. For example, vitamin C may be integrated in a snack for a sun activity with a prolonged duration or typically associated with lowered immunity, such as those that may cause a participant to sleep less or involve exposure to many people, such as spectators in a sports arena.

In some embodiments, the sun defense nutriment may comprise lycopene, which may be found in fruits and vegetable, like tomatoes, watermelon, or papaya. Unlike many other sources of sun defense nutriments, cooking of tomatoes may increase bioavailability of lycopene. Accordingly, tomato products that include cooked tomatoes or a sun defense nutriment that may be used in cooking may be particularly effective. In some aspects, lycopene may be useful as an antioxidant, in cancer prevention, cardiovascular health by reducing LDL cholesterol and lowering blood pressure.

In some embodiments, the sun defense nutriment may comprise vitamin D, which may increase intestinal absorption of critical nutrients such as calcium, iron, magnesium, phosphate, zinc. Vitamin D may be useful in supporting bone health, which may be useful in sun activities that are high intensity and put stress on the participant's skeletal system, such as high impact sports.

In some embodiments, the sun defense nutriment may comprise curcumin, which may be found in turmeric or ginger, for example. Curcumin may have an anti-inflammatory effect, which may be useful for sun activities that may cause inflammation, such as high intensity sports or events with prolonged exposure to heat. In some aspects, the inflammatory sun defense attribute may be combined with the support of bone health sun defense attribute for a snack for a sun activity that may include high impact, high intensity sports with prolonged exposure to heat, such as an intramural or tennis tournament or tailgating with football. For example, the snack may comprise a chew, and the sun defense nutriments may comprise candied ginger (curcumin) and fatty fish such as salmon (Vitamin D).

In some embodiments, the sun defense nutriment may comprise super-fruits such as, for example, Acai Berry, Gogi and Nonie fruits, Mangosteen, Pomegranate and Coffee Berry, which may rank among the most powerful using the natural antioxidant grading system. In some embodiments, the sun defense nutriment may comprise white and green teas, which may provide energy and antioxidant sun defense attributes.

In some embodiments, the sun defense nutriment may comprise astaxanthin, which may be a potent antioxidant carotenoid approximately 1,000 times stronger than vitamin E. Astaxanthin may be found in an orange pigment from microalgae. For example, astaxanthin may be integrated with snacks to provide an orange coloring, such as to complement a citrus flavor that may separately provide Vitamin C.

In some embodiments, the sun defense nutriment may comprise ergothioneine, which may act as an antioxidant and may be superior to CoQ10 and Idebenone. This amino acid may be found in mushrooms and may be crucial for energizing antioxidants in cells.

In some embodiments, the sun defense nutriment may comprise Spirulina, which may act as an antioxidant due to its high levels of beta carotene. Spirulina is a biomass of cyanobacteria and may generally come in the form of a flake, powder, or tablet form. Spirulina also contains significant amounts of iron, calcium, niacin, potassium, magnesium, and B vitamins.

In some embodiments, the sun defense nutriment may comprise garden sprout extract or detoxophane. In some aspects, detoxophane may support the skin's natural detoxifying enzymes, enhancing cellular resistance to and the elimination of environmental toxins. In some implementations, garden sprout extract may be useful in sun activities that may expose the participant to harsh environmental pollutants, including, for example, swimming in a chlorinated pool or site-seeing in a high-smog area. As an illustrative example, swim meets typically take place over hours with some athletes participating in events throughout the day. A swimmer may consume a snack bar between heats or events, and the snack bar may comprise garden sprout extract (to increase elimination of environmental toxins), spirulina (to increase recovery), sunflower seeds (with Vitamin D to increase energy), and peanut butter (with protein and Vitamin B).

In some embodiments, the sun defense nutriment may comprise other natural ingredients, such as pear, hibiscus, or Assafoetida, which may protect from sunlight-derived free radicals that damage DNA and may be at the root of wrinkle formation, inflammation and skin sensitivity.

In some embodiments, the sun defense nutriment may comprise sunflower or safflower, whose oils may be rich in Omega-6, which may keep skin soft, hydrated, and calm by fighting off free radicals that destroy skin's natural moisture barrier.

In some embodiments, the sun defense nutriment may comprise baobab, which may be used as a dried fruit powder, thickener, or sweetener. Baobab may be an effective source of fiber, calcium, magnesium, potassium, iron, and vitamin C. In some aspects, baobab may be integrated into snacks as formation components, such as a base for ice cream, added to fermenting process for beer.

In some embodiments, the sun defense nutriment may comprise garlic, wherein the scent may be released through pores after ingestion, such as through perspiration. The released garlic may be useful as insect repellant. Accordingly, garlic may be integrated into sun defense nutriments for sun activities that may expose participants to insects, such as fishing, camping, hiking, or tailgating.

As an illustrative example, the sun activity may comprise an Iron Man competition, which puts the body through prolonged and extreme exercise conditions. Participants may be susceptible to injury, skin damage, dehydration, lowered immunity after the race, exhaustion, and reduced energy, as non-limiting examples. A snack may comprise sun defense nutriments that protect against one or more of those sun activity side effects. A snack may comprise a gummy with an electrolyte water base (to protect against dehydration), green tea (energy to protect against exhaustion), orange extract with Vitamin C (to protect against lowered immunity and skin damage), CoQ10 microspheres (to protect against muscle breakdown), and ginger (to protect against inflammation). The gummy form may allow for increased bioavailability, which may be useful for the participant to consume throughout the race.

As an illustrative example, the sun activity identified at 605 may be a marathon wherein a portion of the run occurs in high altitudes in the snow. In evaluating a marathon at 610, the sun activity side effects may include dehydration, extended exposure to sun, skin drying from extended exposure to cold, muscle fatigue, loss of energy, and a slowed digestion from the exertion in high altitudes. Some of the side effects may be associated with sun defense attributes at 615, and some of the side effects may be considered factors in identifying the type of sun defense nutriment at 625.

Dehydration may be combated with hydration and dehydration prevention, such as by integrating strawberries, bell peppers, watermelons, or cucumbers into a liquid snack, which may rehydrate and limit dehydration. Extended exposure to sun may be combated with sun protection, wherein a snack may comprise blueberries, nuts, seeds, watermelon, carrots, cauliflower, or green tea. Skin drying may be combated with skin hydration. Muscle fatigue may be combated with inflammation reduction and pain relief. Loss of energy may be combated with energy. Slowed digestion may be combated with metabolic enhancers.

Characteristics of the sun defense nutriment may be influenced by one or more of the sun activity side effects and the sun activity itself. For example, a runner may need sun defense nutriments that are portable and easy to consume, such as gel packets, bite-sized portions, or fluids. Slowed digestion, caused by the high altitudes, may be combated with an easily digestible type of sun defense nutriment, such as liquids or quick-dissolving foods, which may also allow for a quick delivery of energy. Accordingly, the types of sun defense nutriments selected at 625 may include gel packets, drink shots, or bite-sized chewable candy.

Runners commonly crave both sweet and salty flavors, and the flavor profile selected at 625 may reflect that craving. Similarly, where the sun defense attributes include increased rate of metabolism and immediate energy, a complementary flavor profile may be espresso, as it may generally be associated with high amounts of caffeine. Accordingly, the type and flavor profile selected at 625 may be a gel packet with a flavor of mocha espresso with sea salt.

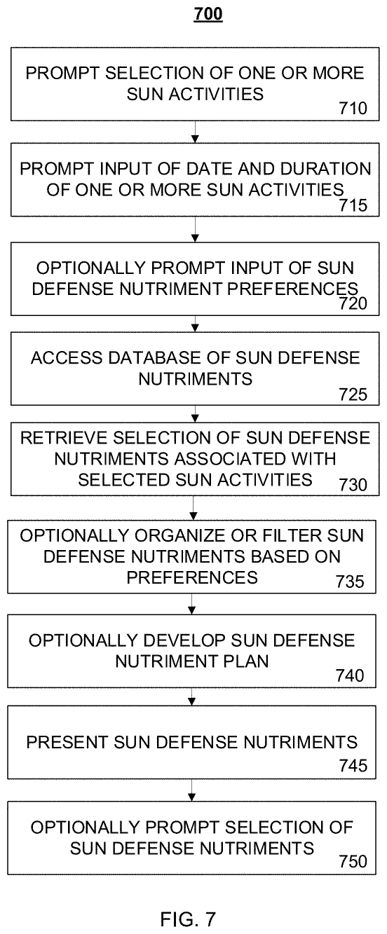

Referring now to FIG. 7, an exemplary method 700 for providing sun defense nutriments for an event with one or more sun activities is illustrated. At 710, selection of one or more sun activities may be prompted. At 715, input of date and duration of the one or more sun activities may be prompted. At 720, input of sun defense nutriment preferences may optionally be prompted. At 725, a database of sun defense nutriments may be accessed or consulted. At 730, a selection of sun defense nutriments associated with the selected sun activities may be retrieved. At 735, the retrieved sun defense nutriments may optionally be organized or filtered based on the received sun defense nutriment preferences. At 740, a sun defense nutriment plan may be developed utilizing one or more of the retrieved sun defense nutriments. At 745, the retrieved sun defense nutriments may be presented. At 750, selection of one or more of the presented sun defense nutriments may be prompted.

In some embodiments, a participant may want to develop a sun activity defense plan for an anticipated sun activity, and a sun defense nutriment planner may prompt participant to input a date and duration of the anticipated sun activity. This may allow the sun defense nutriment planner to offer pre-activity sun defense nutriments that may provide the participant with sustained protection.

As an illustrative example, a participant may be preparing for a three-day fishing trip that will occur in a week. The participant may select multiple sun activities that may occur throughout the fishing trip, as a fishing trip may include fishing, swimming, hiking, and barbecuing. The nutriment planner may suggest a variety of sun defense nutriments that may be consumed in preparation for the fishing trip, throughout the fishing trip, and after the fishing trip.

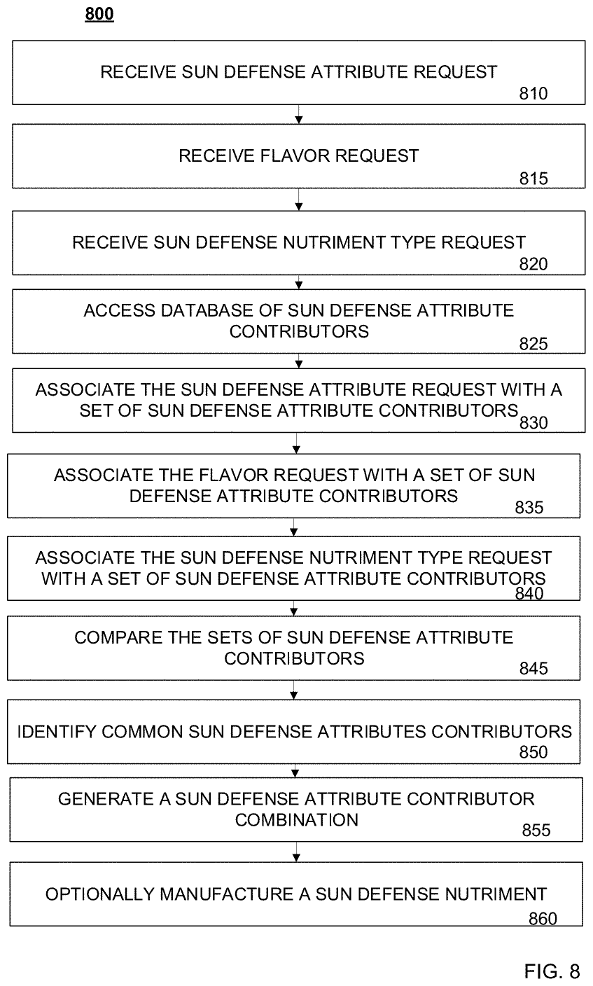

Referring now to FIG. 8, exemplary method steps for developing a sun defense nutriment are illustrated. At 810, a sun defense attribute request may be received. At 815, a flavor request may be received. At 820, a sun defense nutriment type request may be received. At 825, a database of sun defense nutriments may be accessed. At 830, the sun defense attribute request may be associated with a set of sun defense nutriments. At 835, the flavor request may be associated with a set of sun defense nutriments. At 840, the sun defense nutriment type may be associated with a set of sun defense nutriments.

At 845, the sets of sun defense nutriments may be compared, and at 850, one or more common sets of sun defense nutriments may be identified. At 855, one or more sun defense nutriment combinations may be generated. At 860, a sun defense nutriment may be manufactured that fulfills one or more of the sun defense attribute requests, flavor requests, and sun defense nutriment type request.

As an illustrative example, a user may be preparing for a boating trip and may request a sun nutriment in the form of a bar that has chocolate and nut flavors to protect against sun exposure and sea sickness. Different sets of sun defense nutriments may be associated with each request. For example, chocolate flavors may include sun defense nutriments that would not be suitable for a bar, such as any in a liquid form or any requiring refrigeration. Sun defense nutriments that may be suitable for bar may include flavors other than chocolate and berry and may provide protection against risks other than sun exposure and insects. The sets of sun defense nutriments may be compared and common sets may be identified.

For example, one set may include chocolate chips, peanuts, ginger, cherries, and oats. The chocolate chips and peanuts may provide the requested chocolate and nut flavors. The peanuts and cherries may provide sun defense through Vitamins E and C. The ginger may mitigate sea sickness, and the oats may provide adequate binding to create a bar.

As another illustrative example, a user may be preparing for a camping trip and may request a savory additive for potatoes that may protect against sun exposure, insects, and dehydration. A possible set of sun defense nutriments may comprise garlic, cayenne pepper, chili pepper, almond oil, and salt, wherein all of the sun defense nutriments provide a savory flavor. The almond oil may be a convenient additive that may be drizzled over cooked potatoes or may be used during the cooking process. The cayenne and chili peppers may provide Vitamin C, and the almond oil may provide Vitamin E, which may provide sun defense. The garlic may be useful to protect against insects, and the salt may provide a sodium balance to the potassium found in potatoes, which may help with dehydration.

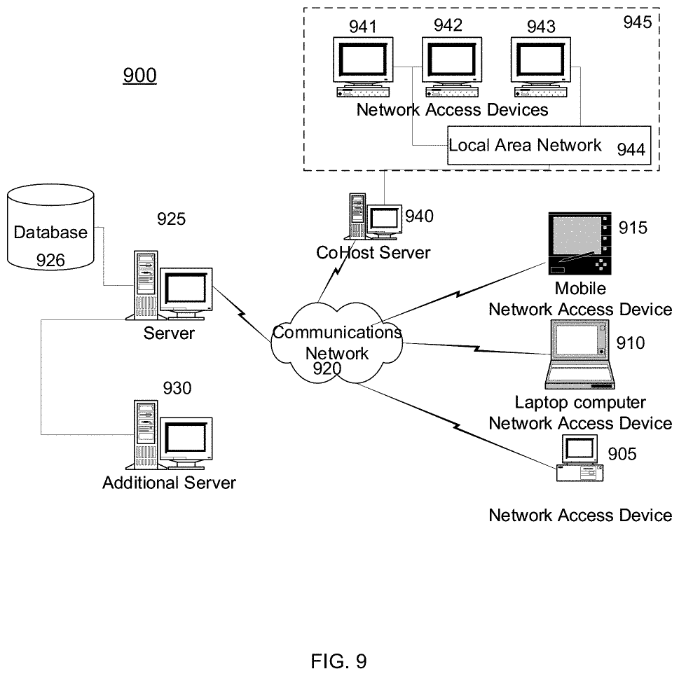

Referring now to FIG. 9, an exemplary processing and interface system 900 is illustrated. In some aspects, access devices 915, 910, 905, such as a mobile device 915 or laptop computer 910 may be able to communicate with an external server 925 though a communications network 920. The external server 925 may be in logical communication with a database 926, which may comprise data related to identification information and associated profile information. In some examples, the server 925 may be in logical communication with an additional server 930, which may comprise supplemental processing capabilities.

In some aspects, the server 925 and access devices 905, 910, 915 may be able to communicate with a co-host server 940 through a communications network 920. The co-host server 940 may be in logical communication with an internal network 945 comprising network access devices 941, 942, 943 and a local area network 944. For example, the co-host server 940 may comprise a payment service, such as PayPal or a social network, such as Facebook or Twitter.

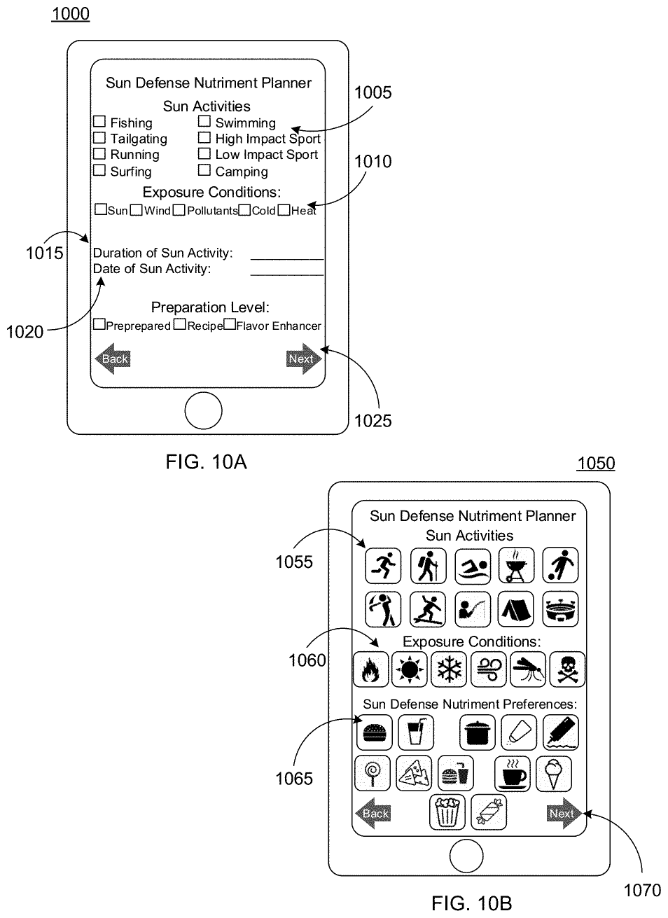

Referring now to FIG. 10A, an exemplary application interface 1000 for a sun defense nutriment planner is illustrated, wherein the prompts 1005, 1010, 1015, 1020 may be presented as words that a participant may select or fill in, as appropriate. In some aspects, the sun defense nutriment planner may comprise a sun activity prompt 1005, which may allow for selection of one or more sun activity. In some implementations, the sun defense nutriment planner may comprise an exposure condition prompt 1010, which may allow for selection of one or more conditions that a participant may expect to be exposed to.

In some aspects, the sun defense nutriment planner may comprise a duration and date prompt 1015, which may allow for the input of the anticipated start date and duration of the one or more selected sun activities. In some implementations, the sun defense nutriment planner may comprise a preparation level prompt 1020, which may allow selection of the intensity of preparation that a participant may desire in the sun defense nutriments. The sun defense nutriment planner may comprise an action prompt 1025 that may allow for control of the progress.

Referring now to FIG. 10B, another exemplary application interface 1050 for a sun defense nutriment planner is illustrated, wherein the prompts 1055, 1060, 1065 may be presented as icons that a participant may select, as appropriate. In some aspects, the sun defense nutriment planner may comprise a sun activity prompt 1055, which may allow for selection of one or more sun activity, including, for example, running, hiking, swimming, grilling, high impact sport, low impact sport, surfing, fishing, camping, or sport spectating.

In some implementations, the sun defense nutriment planner may comprise an exposure condition prompt 1060, which may allow for selection of one or more conditions that a participant may expect to be exposed to, including, for example, heat, sunlight, cold, wind, insects, or pollutants. In some aspects, the sun defense nutriment planner may comprise a sun defense nutriment preference prompt 1065, which may allow for the input of one or more preferences for the sun defense nutriments, including, for example, salty or sweet; drink or food; recipe component, additive, or condiment; small bite, snack, or meal; or hot or cold. The sun defense nutriment planner may comprise an action prompt 1070 that may allow for control of the progress.

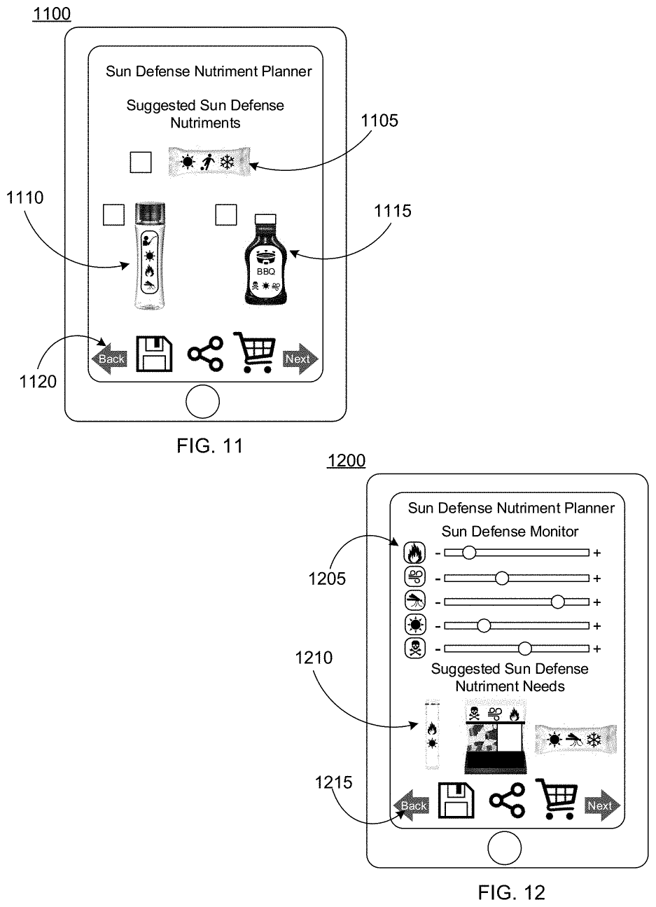

Referring now to FIG. 11, an exemplary application interface 1100 for a sun defense nutriment planner is illustrated, wherein the resulting suggested sun defense nutriments 1105, 1110, 1115 may be presented. In some aspects, the sun defense nutriment planner may suggest a nutrient bar 1105 for a high impact sport that may further provide protection against sunlight and cold. In some embodiments, the sun defense nutriment planner may suggest a beverage enhancer 1110 that may be appropriate for fishing that may further provide protection against sunlight, heat, and insects. The sun defense nutriment planner may suggest a barbecue sauce 1115 that may be appropriate for watching a sport, such as soccer or football, which may further offer protection against pollutants, sunlight, and wind.

In this illustrative example, a participant may be preparing for a fishing and sports trip where the sporting activities may take place in high altitudes, such as in the mountains, and the fishing may occur in a nearby lake, which may be far from the mountains. In some aspects, the sun defense nutriment planner may comprise action prompts 1120, which may allow for control of the progress of the planning and for direct responses to the suggested sun defense nutriments 1105, 1110, 1115. For example, the suggested sun defense nutriments 1105, 1110, 1115 may be saved for reference later, shared on social media, or added to cart for purchase.

Referring now to FIG. 12, an exemplary application interface 1200 for a sun defense nutriment planner is illustrated, wherein sun defense monitors may indicate sun defense levels 1205 in a participant. The application interface 1200 may provide sun defense nutriment suggestions 1210 based on the sun defense levels 1205. In some aspects, the sun defense levels 1205 may be determined by monitoring levels of particular sun defense nutriments. In some aspects, the sun defense levels 1205 may be determined by average digestion rates associated with ingested sun defense nutriments.

In some aspects, the sun defense levels 1205 may be based on data from multiple sources, such as, for example, wearable technology such as Fitbits or heart monitors, typical metabolism of purchased, selected, or input sun defense nutriments. In some embodiments, the sun defense nutriment planner application interface 1200 may comprise action prompts 1215, which may allow a participant to toggle between screens or save, share, or purchase the suggested sun defense nutriments 1210.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order or that all illustrated operations be performed to achieve desirable results. In certain circumstances, multi-tasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multi-tasking and parallel processing may be advantageous. Nevertheless, it will be understood that various modifications may be made without departing from the spirit and scope of the claimed disclosure.

What is claimed is:

1. A snack with sun defense nutriments comprising
a first sun defense nutriment comprising a first sun defense attribute and a first predefined flavor; and
a second sun defense nutriment comprising a second sun defense attribute and a second predefined flavor, wherein, as the snack is ingested, release of the first sun defense attribute and the second sun defense attribute initiates protection against one or more sun activity side effects associated with a predefined sun activity.

2. The snack of claim 1, wherein the snack comprises a bar.

3. The snack of claim 1, wherein the snack comprises a chew.

4. The snack of claim 1, wherein the snack comprises other ingredients that do not comprise sun defense attributes.

5. The snack of claim 1, wherein the first sun defense attribute comprises a water-soluble antioxidant, fat-soluble antioxidant, or a combination thereof.

6. The snack of claim 1, wherein at least one of the one or more sun activity side effects comprises sun exposure.

7. The snack of claim 6, wherein one or both the first sun defense attribute and the second sun defense attribute comprises Vitamin C, Vitamin E, or both.

8. The snack of claim 6, wherein one or both the first sun defense nutriment and second sun defense nutriment comprise at least one of: chlorella, peanut butter, spirulina, cherries.

9. The snack of claim 1, wherein the predefined sun activity causes exposure to sunlight, and at least one of the one or more sun activity sun side effects comprises skin damage.

10. The snack of claim 9, wherein one or both the first sun defense attribute and the second sun defense attribute protects against skin damage.

11. The snack of claim 10, wherein one or both the first sun defense nutriment and the second sun defense nutriment comprise at least one of: dark chocolate, cherries, chlorella, green tea, spirulina, flax seeds, and chia seeds.

12. A snack with sun defense nutriments comprising:
a plurality of sun defense nutriments each comprising at least one predefined sun defense attribute and a predefined flavor, wherein, as the snack is ingested, release of the at least one predefined sun defense attribute initiates protection against a one or more sun activity side effects associated with a predefined sun activity.

13. The snack of claim 12, wherein the snack only comprises the plurality of sun defense nutriments.

14. The snack of claim 12, wherein the snack further comprises other ingredients that do not comprise sun defense attributes.

15. The snack of claim 12, wherein the plurality of sun defense nutriments comprises a combination of at least one seed type and at least one nut butter type.

16. The snack of claim 12, wherein the snack comprises a bar.

17. The snack of claim 12, wherein the snack comprises a chew.

18. The snack of claim 12, wherein the one or more sun activity side effects comprise any combination of skin damage, dehydration, insect bites, or overheating.

19. The snack of claim 18, wherein at least one of the plurality of sun defense nutriments comprises garlic, and wherein at least one of the one or more sun activity side effects comprise insect bites.

20. The snack of claim 18, wherein at least a portion of sun defense attributes from a plurality of the at least one predefined sun defense attributes from the sun defense nutriments comprise any combination of Vitamin A, Vitamin C, Vitamin E, Vitamin D, Vitamin B, potassium, or electrolytes.
